(19) United States
(12) Patent Application Publication
(10) Pub. No.: US 2025/0049967 A1
(43) Pub. Date: Feb. 13, 2025

(54) PET TRACERS FOR VISUALIZING GABAA GAMMA1 RECEPTORS

(71) Applicant: Hoffmann-La Roche Inc., Little Falls, NJ (US)

(72) Inventors: Giuseppe CECERE, Basel (CH); Luca Claudio GOBBI, Buus (CH); Maria-Clemencia HERNANDEZ, Delémont (CH); Michael Carl HONER, Zürich (CH)

(73) Assignee: Hoffmann-La Roche Inc., Little Falls, NJ (US)

(21) Appl. No.: 18/933,079

(22) Filed: Oct. 31, 2024

Related U.S. Application Data
(63) Continuation of application No. PCT/EP2023/061439, filed on May 2, 2023.

(30) Foreign Application Priority Data
May 3, 2022 (EP) 22171249.0

(51) Int. Cl. A61K 51/04; C07B 59/00

(52) U.S. Cl. CPC A61K 51/047; C07B 59/002; A61K 2123/00; C07B 2200/05

(57) ABSTRACT

The present invention provides radiolabeled GABA_{A} γ1 positive allosteric modulators (PAM) that are useful for medical imaging.

### CROSS REFERENCE TO RELATED APPLICATIONS

This Application is a continuation of International Application No. PCT/EP2023/061439 filed on May 2, 2023, which claims priority to EP Application No. 22171249.0 filed on May 3, 2022, the disclosures of which are incorporated herein by reference.

### FIELD OF THE INVENTION

The invention relates to radiolabeled GABA_{A} γ1 positive allosteric modulators (PAM) useful for medical imaging, such as positron-emission tomography (PET) and/or autoradiography.

### BACKGROUND OF THE INVENTION

Receptors for the major inhibitory neurotransmitter, gamma-aminobutyric acid (GABA), are divided into two main classes: (1) GABA_{A} receptors, which are members of the ligand-gated ion channel superfamily and (2) GABA_{B} receptors, which are members of the G-protein linked receptor family. The GABA_{A} receptor complex which is a membrane-bound heteropentameric protein polymer is composed principally of α, β and γ subunits. GABA_{A} receptors are ligand-gated chloride channels and the principal mediators of inhibitory neurotransmission in the human brain.

There are 19 genes encoding for GABA_{A} receptor subunits that assemble as pentamers with the most common stoichiometry being two α, two β and one γ subunit. GABA_{A} subunit combinations give rise to functional, circuit, and behavioral specificity, and their expression is distributed heterogeneously within the brain. The GABA_{A} γ1 subunit-containing receptors are less abundant (around 5-10% of total expression of GABA_{A} receptors in the brain) compared to those containing the γ2 subunit.

Positron emission tomography (PET), as a non-invasive imaging technique, is supporting drug discovery and development by providing valuable information on drug-target engagement, accessing drug occupancy and monitoring treatment. Moreover, PET can be used for neuroreceptor mapping in healthy subjects and diseased states (Nasrallah I, Dubroff, J An overview of PET neuroimaging, Seminars in nuclear medicine, 2013, 43, 449-61. Hou L, Rong J, Haider A, et al. Positron Emission Tomography Imaging of the Endocannabinoid System: Opportunities and Challenges in Radiotracer Development, J. Med. Chem. 2021, 64, 1, 123-149). Alternatively, autoradiography can be used for the in vitro neuroreceptor mapping on tissue sections obtained post mortem. In animal models, autoradiography can provide drug-target engagement information from an ex vivo readout. PET imaging of GABA_{A} γ2 subunit-containing receptors is well established, with a number of radiotracers routinely used in pre-clinical and clinical studies. Most prominent and important among these are [¹⁸F]Flumazenil (or the carbon-11 version [¹¹C]Flumazenil) and [¹¹C]Ro15-4513 (Kassenbrock A, Vasdev N, Liang S H, Selected PET Radioligands for Ion Channel Linked Neuroreceptor Imaging: Focus on GABA, NMDA and nACh Receptors, Current Topics in Medicinal Chemistry, 2016, 16, 1830-1842). [¹⁸F]Flumazenil is visualizing GABA_{A} γ2 receptors containing all combinations of a subunits, while [¹¹C]Ro15-4513 is preferring GABA_{A} α5γ2 receptors. In contrast to GABA_{A} γ2 receptors. While selective GABA_{A} γ1 tracers for in vitro applications have been described (see, e.g., WO2021198124 and WO2021213952), no PET tracers exist targeting and selectively visualizing GABA_{A} γ1 receptors in vivo. Therefore, there is an unmet need for such PET tracers. Compounds of the present invention display increased affinity towards GABA_{A} γ1 receptors and allow for imaging of the target in vivo.

### SUMMARY OF THE INVENTION

The radiolabeled compounds of the invention are selective GABA_{A} γ1 receptor positive allosteric modulators (PAM), useful to visualize this receptor in vivo and in vitro, e.g. by PET or autoradiography. The compounds of the present invention have high binding affinity and selectivity for the γ1-containing subtypes (α5γ1, α2γ1, α1γ1) relative to the γ2-containing subtypes (e.g. α1γ2, α2γ2, α3γ2 and α5γ2). As such, compounds of the present invention are useful to visualize GABA_{A} γ1 receptors that are not addressed by classical GABA_{A} receptor tracers.

In a first aspect, the present invention provides a compound of formula (I) or (II)

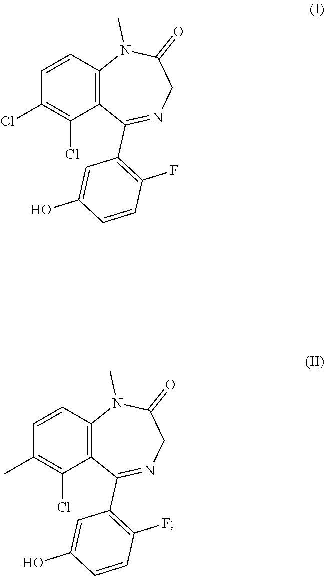 [SMILES: CN1C(=O)CN=C(c2cc(O)ccc2F)c2c1ccc(Cl)c2Cl ; Cc1ccc2c(c1Cl)C(c1cc(O)ccc1F)=NCC(=O)N2C]

or a pharmaceutically acceptable salt thereof, wherein said compound comprises a radiolabel.

In a further aspect, the present invention provides a radiolabeled compound described herein for use in GABA_{A} γ1 occupancy studies.

In a further aspect, the present invention provides a radiolabeled compound described herein for use in diagnostic imaging of GABA_{A} γ1 in a mammal.

### BRIEF DESCRIPTION OF THE DRAWINGS

FIG. 1 shows in vitro autoradiograms of [³H]-(I) and [³H]-(II) in coronal mouse brain sections (refer to Example 5 for details).

FIG. 2 shows line plots of regional time-activity curves (TACs) of [¹¹C]-(I) and [¹¹C]-(II) in baboons (refer to Example 6 for details).

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition, these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like.

The term “mammal” includes humans, non-human primates such as chimpanzees and other apes and monkey species, farm animals such as cattle, horses, sheep, goats, and swine, domestic animals such as rabbits, dogs, and cats, laboratory animals including rodents, such as rats, mice, and guinea pigs. In certain embodiments, a mammal is a human. The term mammal does not denote a particular age or sex.

The term “radiolabel” refers to radioactive isotopes of, e.g., hydrogen, carbon, nitrogen, oxygen, fluorine, and chlorine. In some embodiments, the radiolabels used in the context of the invention are useful for PET imaging and/or autoradiography. Examples of isotopes that can be incorporated into the compounds of formula (I) and (II) as radiolabels include ³H, ¹¹C, ¹⁴C, ¹³N, ¹⁵O, ¹⁸F, and ³⁶Cl, respectively. Preferred radiolabels are ³H, ¹¹C, ¹³N, ¹⁵O, and ¹⁸F. Further preferred radiolabels are ³H, ¹¹C and ¹⁸F. Particularly preferred radiolabels are ³H and ¹¹C.

### Compounds of the Invention

In a first aspect, the present invention provides a compound of formula (I) or (II)

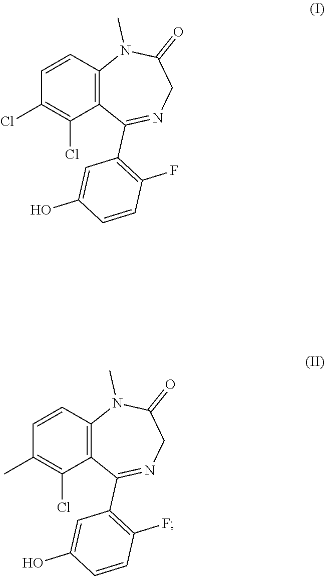 [SMILES: CN1C(=O)CN=C(c2cc(O)ccc2F)c2c1ccc(Cl)c2Cl ; Cc1ccc2c(c1Cl)C(c1cc(O)ccc1F)=NCC(=O)N2C]

or a pharmaceutically acceptable salt thereof, wherein said compound comprises a radiolabel.

The compounds of formula (I) and (II) of the invention are radiolabeled (i.e., isotopically-labeled) by having one or more atoms therein replaced by isotopes having a different atomic mass or mass number. Examples of isotopes that can be incorporated into the compounds of formula (I) and (II) include isotopes of hydrogen, carbon, nitrogen, oxygen, fluorine, and chlorine, such as, but not limited to ³H, ¹¹C, ¹⁴C, ¹³N, ¹⁵O, ¹⁸F, and ³⁶Cl, respectively. Certain isotopically-labeled compounds of formula (I) and (II), for example, those incorporating a radioactive isotope, are useful in drug and/or substrate tissue distribution studies. The radioactive isotopes tritium, i.e., ³H, carbon-11, i.e., ¹¹C and fluorine-18, i.e., ¹⁸F are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with positron emitting isotopes, such as ¹¹C, ¹⁸F, ¹⁵O and ¹³N, can be useful in Positron Emission Topography (PET) studies, e.g. for examining substrate receptor occupancy.

Substitution with beta minus radiation emitting isotopes, such as ³H, can be useful in autoradiography studies, e.g. for examining substrate receptor occupancy.

In one embodiment, said radiolabel is selected from ³H, ¹¹C, ¹³N, ¹⁵O, ¹⁸F, and ³⁶Cl.

In a preferred embodiment, said radiolabel is selected from ¹¹C, ¹⁸F and ³H.

In a preferred embodiment, said radiolabel is selected from ¹¹C and ³H.

In a particularly preferred embodiment, said radiolabel is ¹¹C.

In a particularly preferred embodiment, said radiolabel is ³H.

In a particularly preferred embodiment, said radiolabel is ¹⁸F.

In one embodiment, the compound of formula (I) or (II) of the invention is selected from the group consisting of

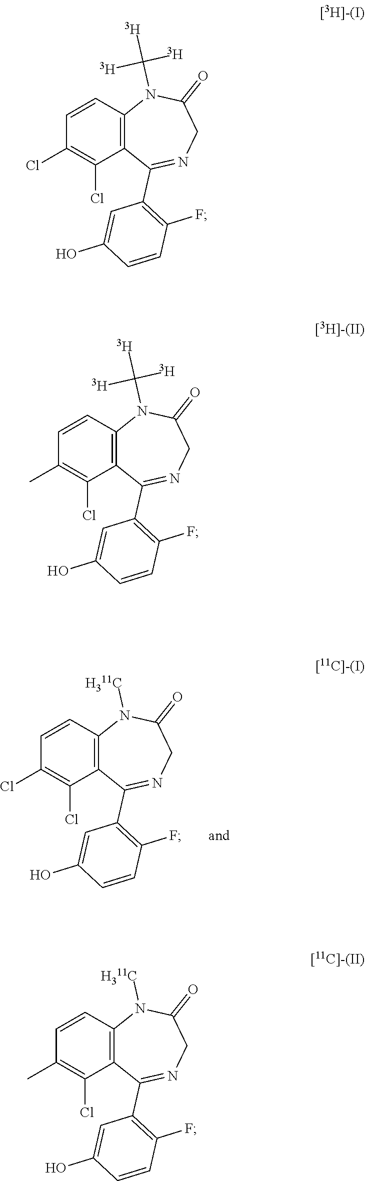 [SMILES: [3H]C([3H])([3H])N1C(=O)CN=C(c2cc(O)ccc2F)c2c1ccc(Cl)c2Cl ; [3H]C([3H])([3H])N1C(=O)CN=C(c2cc(O)ccc2F)c2c1ccc(C)c2Cl ; [11CH3]N1C(=O)CN=C(c2cc(O)ccc2F)c2c1ccc(Cl)c2Cl ; Cc1ccc2c(c1Cl)C(c1cc(O)ccc1F)=NCC(=O)N2[11CH3]]

or a pharmaceutically acceptable salt thereof.

In one embodiment, the compound of formula (I) or (II) of the invention is

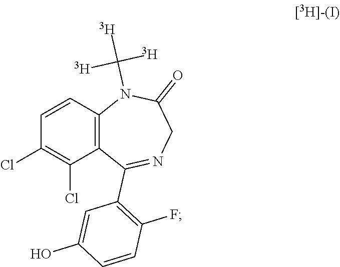 [SMILES: [3H]C([3H])([3H])N1C(=O)CN=C(c2cc(O)ccc2F)c2c1ccc(Cl)c2Cl]

or a pharmaceutically acceptable salt thereof.

In one embodiment, the compound of formula (I) or (II) of the invention is

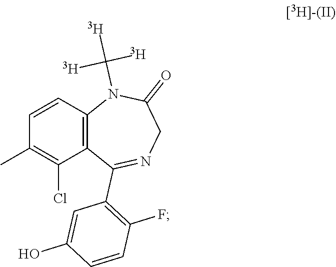 [SMILES: [3H]C([3H])([3H])N1C(=O)CN=C(c2cc(O)ccc2F)c2c1ccc(C)c2Cl]

or a pharmaceutically acceptable salt thereof.

In one embodiment, the compound of formula (I) or (II) of the invention is

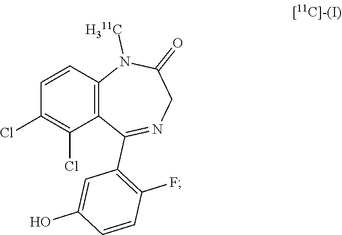 [SMILES: [11CH3]N1C(=O)CN=C(c2cc(O)ccc2F)c2c1ccc(Cl)c2Cl]

or a pharmaceutically acceptable salt thereof.

In one embodiment, the compound of formula (I) or (II) of the invention is

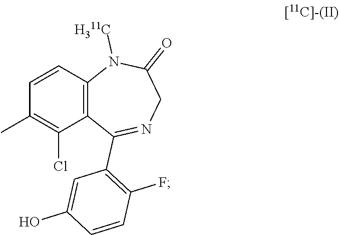 [SMILES: Cc1ccc2c(c1Cl)C(c1cc(O)ccc1F)=NCC(=O)N2[11CH3]]

or a pharmaceutically acceptable salt thereof.

### Using the Compounds of the Invention

The radiolabeled compounds of the present invention are potent GABA_{A} γ1 positive allosteric modulators (PAM) that may be used, for example, as PET tracers for the GABA_{A} γ1 receptor to validate target engagement of therapeutic GABA_{A} γ1 modulators, as well as to investigate the function of the GABA_{A} γ1 receptor under normal and disease conditions.

Thus, in one aspect, the present invention provides a method of diagnostic imaging of GABA_{A} γ1 in a mammal, comprising:
(a) administering to the mammal a detectable quantity of a radiolabeled compound described herein, or of a pharmaceutically acceptable salt thereof; and
(b) detecting the radiolabeled compound when associated with GABA_{A} γ1.

In a preferred embodiment, said diagnostic imaging is diagnostic imaging of the brain.

In a preferred embodiment, said detecting is detecting via autoradiography and/or PET.

In a preferred embodiment, said detecting is detecting via autoradiography.

In a preferred embodiment, said detecting is detecting via PET.

In a further aspect, the present invention provides a radiolabeled compound as described herein, or a pharmaceutically acceptable salt thereof, for use in a method of diagnostic imaging described herein.

In a further aspect, the present invention provides the use of a radiolabeled compound as described herein, or a pharmaceutically acceptable salt thereof, in a method of diagnostic imaging described herein.

In a further aspect, the present invention provides a radiolabeled compound as described herein, or a pharmaceutically acceptable salt thereof, for use in GABA_{A} γ1 occupancy studies. Such occupancy studies may be conducted, for example, as described in Scientific Reports (2021), 11(1), 7700.

In a further aspect, the present invention provides the use of the radiolabeled compound disclosed herein in GABA_{A} γ1 occupancy studies.

In one embodiment, said GABA_{A} γ1 occupancy studies comprise contacting GABA_{A} γ1 with a radiolabeled compound disclosed herein, or a pharmaceutically acceptable salt thereof.

In a further aspect, the present invention provides a method for studying the occupancy of GABA_{A} γ1 receptors, said method comprising contacting said GABA_{A} γ1 receptors with a radiolabeled compound described herein.

In one embodiment, said GABA_{A} γ1 occupancy studies are in vitro occupancy studies.

### EXAMPLES

The invention will be more fully understood by reference to the following examples. The claims should not, however, be construed as limited to the scope of the examples.

All reaction examples and intermediates were prepared under an argon atmosphere if not specified otherwise.

### Building Block Syntheses

The building blocks can be produced according to the following synthetic procedures.

### Building Block A

### 6,7-dichloro-5-(2-fluoro-5-methoxy-phenyl)-1,3-dihydro-1,4-benzodiazepin-2-one

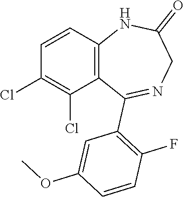 [SMILES: COc1ccc(F)c(C2=NCC(=O)Nc3ccc(Cl)c(Cl)c32)c1]

### a) 5-chloro-2-methyl-3,1-benzoxazin-4-one

A solution of 2-amino-6-chlorobenzoic acid (15.0 g, 87.4 mmol) in acetic anhydride (200 mL) was stirred at 140° C. for 2 h. The reaction solution was concentrated in vacuo. The residue was suspended in acetonitrile, the solid was filtered and the filter cake was dried in in vacuo to afford the title compound (11.3 g, 66%) as a white solid. ¹H NMR (400 MHz, CDCl₃) δ ppm 2.47 (3 H, s) 7.47 (1 H, dd, J=8.1, 0.9 Hz) 7.53 (1 H, dd, J=7.9, 1.0 Hz) 7.67 (1 H, dd, J=8.1, 8.0 Hz).

### b) N-[3-chloro-2-(2-fluoro-5-methoxy-benzoyl)phenyl]acetamide

To a solution of 2-bromo-1-fluoro-4-methoxybenzene (5.45 g, 26.6 mmol) in THF (200 mL) was added n-butyllithium (2.5 M in hexane, 12.8 mL, 31.9 mmol) at −78° C. After stirring for 1 h, 5-chloro-2-methyl-3,1-benzoxazin-4-one (5.20 g, 26.6 mmol) was added to the mixture and stirring was continued for another 1 h at −78° C. The mixture was quenched with aqueous saturated NH₄Cl and extracted with ethyl acetate. The organic layer was dried (Na₂SO₄), filtered and concentrated in vacuo. The residue was purified by preparative HPLC (Phenomenex luna C_{18, 10} μm, 250×50 mm, 0.05% HCl in water/acetonitrile) to afford the title compound (3.63 g, 42%) as a light yellow solid. MS: 322.1 ([{³⁵Cl}M+H]⁺), 324.1 ([{³⁷Cl}M+H]⁺), ESI pos.

### c) (2-amino-6-chloro-phenyl)-(2-fluoro-5-methoxy-phenyl)methanone

To a solution of N-[3-chloro-2-(2-fluoro-5-methoxy-benzoyl)phenyl]acetamide (4.00 g, 12.4 mmol) in ethanol (50 mL) was added aqueous HCl (37%, 53.3 mL, 640 mmol). The mixture was stirred at 100° C. for 2 h and then concentrated in vacuo. The residue was dissolved in DCM and washed with saturated aqueous NaHCO₃ and water successively. The organic layer was dried (Na₂SO₄), filtered and concentrated in vacuo to afford the title compound (2.87 g, 83%) as an off-white solid. MS: 280.0 ([{³⁵Cl}M+H]⁺), 282.0 ([{³⁷Cl}M+H]⁺), ESI pos.

### d) (6-amino-2,3-dichloro-phenyl)-(2-fluoro-5-methoxy-phenyl)methanone

A solution of (2-amino-6-chloro-phenyl)-(2-fluoro-5-methoxy-phenyl)methanone (1.00 g, 3.58 mmol) and N-chlorosuccinimide (430 mg, 3.22 mmol) in DMF (20 mL) was stirred at 0° C. for 2 h. The mixture was quenched with water and extracted with DCM. The organic layer was dried (Na₂SO₄), filtered and concentrated in vacuo. The residue was purified by preparative HPLC (Phenomenex Synergi C18, 10 μm, 150×25 mm, 0.1% trifluoroacetic acid in water/acetonitrile) to afford the title compound (367 mg, 33%) as a light yellow solid. MS: 313.9 ([{³⁵Cl, ³⁵Cl}M+H]⁺), 315.9 ([{³⁵Cl, ³⁷Cl}M+H]⁺), ESI pos.

### e) 6.7-dichloro-5-(2-fluoro-5-methoxy-phenyl)-1,3-dihydro-1,4-benzodiazepin-2-one

A solution of glycine ethyl ester hydrochloride (2.89 g, 20.7 mmol) and (6-amino-2,3-dichloro-phenyl)-(2-fluoro-5-methoxy-phenyl)methanone (650 mg, 2.07 mmol) in pyridine (30 mL) was stirred at 100° C. for 16 h. The mixture was concentrated in vacuo. The residue was purified by preparative HPLC (Phenomenex Synergi C18, 10 μm, 150×25 mm, 0.1% trifluoroacetic acid in water/acetonitrile) to afford the title compound (280 mg, 38%) as a yellow solid. MS: 353.0 ([{³⁵Cl, ³⁵Cl}M+H]⁺), 355.0 ([{³⁵Cl, ³⁷Cl}M+H]⁺), ESI pos.

### Building Block B

### 6-chloro-5-(2-fluoro-5-methoxy-phenyl)-7-methyl-1,3-dihydro-1,4-benzodiazepin-2-one

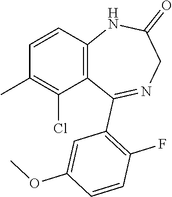 [SMILES: COc1ccc(F)c(C2=NCC(=O)Nc3ccc(C)c(Cl)c32)c1]

### a) (6-amino-3-bromo-2-chloro-phenyl)-(2-fluoro-5-methoxy-phenyl)methanone

In analogy to experiment of Building block A d, (2-amino-6-chloro-phenyl)-(2-fluoro-5-methoxy-phenyl)methanone (Building block A c) using N-bromosuccinimide instead of N-chlorosuccinimide was converted into the title compound (1.63 g, 64%) which was obtained as a light yellow solid. MS: 357.9 ([{⁷⁹Br, ³⁵Cl}M+H]⁺), 359.9 ([{⁸¹Br, ³⁵Cl or ⁷⁹Br, ³⁷Cl}M+H]⁺), ESI pos.

### b) 7-bromo-6-chloro-5-(2-fluoro-5-methoxy-phenyl)-1,3-dihydro-1,4-benzodiazepin-2-one

In analogy to experiment of Building block A e, (6-amino-3-bromo-2-chloro-phenyl)-(2-fluoro-5-methoxy-phenyl)methanone was converted into the title compound (1.63 g, 64%) as a light yellow solid (860 mg, 38%) which was obtained as a light yellow solid. MS: 396.9 ([{⁷⁹Br, ³⁵Cl}M+H]⁺), 398.9 ([{⁸¹Br, ³⁵Cl or ⁷⁹Br, ³⁷Cl}M+H]⁺), ESI pos.

### c) 6-chloro-5-(2-fluoro-5-methoxy-phenyl)-7-methyl-1,3-dihydro-1,4-benzodiazepin-2-one

A solution of 7-bromo-6-chloro-5-(2-fluoro-5-methoxy-phenyl)-1,3-dihydro-1,4-benzodiazepin-2-one (600 mg, 1.51 mmol), methylboronic acid (117 mg, 1.96 mmol), potassium phosphate (641 mg, 3.02 mmol) and [1,1′-bis(diphenylphosphino)ferrocene]dichloropalladium(II) (1.10 g, 1.51 mmol) in DMF (12 mL) was stirred at 80° C. for 6 h under nitrogen. The reaction was diluted with methanol, filtered through a plug of Celite and the filtrate was concentrated in vacuo. The residue was treated with water and extracted with ethyl acetate. The organic layer was dried (Na₂SO₄), filtered and concentrated in vacuo. The residue was purified by preparative HPLC (Phenomenex Synergi C18, 10 μm, 150×25 mm, 0.1% trifluoroacetic acid in water/acetonitrile) to afford the title compound (230 mg, 45%) as a light brown solid. MS: 333.1 ([{⁷⁹Br, ³⁵Cl}M+H]⁺), 335.1 ([{⁸¹Br, ³⁵Cl or ⁷⁹Br, ³⁷Cl }M+H]⁺), ESI pos.

### ¹¹C Radiolabeling Precursor 1

### 5-[5-[tert-butyl(dimethyl)silyl]oxy-2-fluoro-phenyl]-6,7-dichloro-1,3-dihydro-1,4-benzodiazepin-2-one

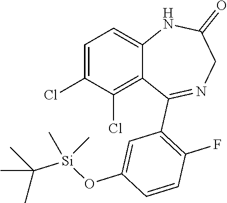 [SMILES: CC(C)(C)[Si](C)(C)Oc1ccc(F)c(C2=NCC(=O)Nc3ccc(Cl)c(Cl)c32)c1]

### a) tert-butyl N-[3,4-dichloro-2-[(2-fluoro-5-methoxy-phenyl)-hydroxy-methyl]phenyl]carbamate

To a solution of tert-butyl N-(3,4-dichlorophenyl)carbamate (5.82 g, 22.2 mmol) in THF (64 mL) was added dropwise from a dry-ice-cooled dropping-funnel at −90° C. tert-butyllithium, 1.7 M in pentane (28.7 ml, 48.8 mmol) and the resulting mixture was stirred at −85° C. for an additional 0.5 h. Then was added dropwise from a dry-ice-cooled dropping-funnel at −85 to −90° C. a solution of 2-fluoro-5-methoxybenzaldehyde (3.76 g, 24.4 mmol) in THF (16 ml). After stirring at −90 to −85° C. for an additional 0.5 h the mixture was allowed to warm to −65° C. and was then quenched by dropwise addition of saturated aqueous NH₄Cl. The mixture was diluted with water and extracted with ethyl acetate. The organic layer was washed with brine, dried over sodium sulfate, filtered and concentrated in vacuo. The residue was suspended in DCM, the solid was filtered and dried in high vacuum to afford the title compound (4.29 g, 46%) as a white solid. MS: 414.2 ([{³⁵Cl, ³⁵Cl}M−H]⁻), 416.2 ([{³⁵Cl, ³⁷Cl}M−H]⁻), ESI neg.

### b) tert-butyl (3,4-dichloro-2-(2-fluoro-5-methoxybenzoyl)phenyl)carbamate

To a suspension of tert-butyl N-[3,4-dichloro-2-[(2-fluoro-5-methoxy-phenyl)-hydroxy-methyl]phenyl]carbamate (9.58 g, 23 mmol) in DCM (95 ml) was added at 22° C. water (95 ml), potassium bromide (383 mg, 3.22 mmol) and sodium bicarbonate (773 mg, 9.21 mmol) to give two layers that were cooled to 0° C. Then TEMPO (75.5 mg, 483 μmol) was added and sodium hypochlorite (21.4 g, 17.8 ml, 34.5 mmol) was added under vigorous stirring dropwise (over 1 h), while internal temperature was kept below 2° C. The mixture was allowed to warm to 22° C. and extracted with DCM (3×100 ml). The organic layers were washed with water (1×100 ml) and half-sat NaCl (1×100 ml), dried over Na₂SO₄, filtered and evaporated. The residue (light brown foam) was treated with EtOAc (50 ml) to give precipitation, the suspension was stirred for 30 minutes and the solid was filtered off, washed with EtOAc (2×20 ml) and dried to give product (5.602 g, 59%) as white solid. The filtrate was concentrated, adsobed on Isolute sorbent and purified by flash chromatography (silica gel, 330 g, adsorbed on Isolute HM-N, EtOAc in heptane 5% to 10%) to give additional product (3.00 g, 31%) as white solid. 412.2 ([{³⁵Cl, ³⁵Cl}M−H]⁻), 414.2 ([{³⁵Cl, ³⁷Cl}M−H]⁻), ESI neg.

### c) (6-amino-2,3-dichloro-phenyl)-(2-fluoro-5-methoxy-phenyl)methanone

To a solution of tert-butyl N-[3,4-dichloro-2-[(2-fluoro-5-methoxy-phenyl)-hydroxy-methyl]phenyl]carbamate (8.60 g, 20.8 mmol) in DCM (200 mL) was added at 22° C. trifluoroacetic acid (47.3 g, 415 mmol) and the mixture was stirred at 22° C. for 2 h. The solution was concentrated in vacuo. The residue (combined with another batch—17.9 mmol-scale) was treated with saturated aqueous NaHCO₃ and extracted with ethyl acetate. The organic layer was dried over magnesium sulfate, filtered and concentrated in vacuo to afford the title compound (11.0 g, 90%) as a yellow solid. MS: 314.0 ([{³⁵Cl, ³⁵Cl}M+H]⁺), 316.0 ([{³⁵Cl, ³⁷Cl}M+H]⁺), ESI pos.

### d) 6,7-dichloro-5-(2-fluoro-5-methoxy-phenyl)-1,3-dihydro-1,4-benzodiazepin-2-one

### Building Block A, Alternative Synthesis

A solution of (6-amino-2,3-dichloro-phenyl)-(2-fluoro-5-methoxy-phenyl)methanone (8.35 g. 26.6 mmol) in pyridine (165 mL) was heated to 90° C., then ethyl glycinate hydrochloride (26.0 g, 186 mmol) was added in one portion, and the resulting mixture was stirred at 110° C. for 4 h. The mixture was cooled to 90° C., then further ethyl glycinate hydrochloride (14.8 g, 106 mmol) was added, and stirring at 110° C. was continued for 16 h. The mixture was cooled to room temperature and concentrated in vacuo. The residue was treated with saturated aqueous NaHCO₃ and extracted with ethyl acetate. The organic layer was washed with brine, dried over sodium sulfate, filtered and concentrated in vacuo. The residue was purified by flash column chromatography (silica, 10-100% ethyl acetate in heptane) to afford the title compound (5.47 g, 58%) as a yellow solid. MS: 353.0 ([{³⁵Cl, ³⁵Cl}M+H]⁺), 355.0 ([{³⁵Cl, ³⁷Cl}M+H]⁺), ESI pos.

### e) 6,7-dichloro-5-(2-fluoro-5-hydroxy-phenyl)-1,3-dihydro-1,4-benzodiazepin-2-one

To a light yellow solution of 6,7-dichloro-5-(2-fluoro-5-methoxy-phenyl)-1,3-dihydro-1,4-benzodiazepin-2-one (500 mg, 1.42 mmol) in DCM (15 mL) was added dropwise at −65° C. boron tribromide (1.77 g, 7.08 mmol). The mixture was allowed to warm to −20° C. and stirred for 0.5 h. The mixture was quenched with half-saturated aqueous NaHCO₃ and extracted with DCM. The organic layer was washed with half-saturated aqueous NaHCO₃, dried over sodium sulfate, filtered and concentrated in vacuo. The aqueous layer was again extracted with ethyl acetate, the organic layer was dried over sodium sulfate, filtered and concentrated in vacuo. Both residues were combined and purified by flash column chromatography (silica, 10-100% ethyl acetate in heptane) followed by crystallization from MTBE/heptane to afford the title compound (220 mg, 46%) as a light yellow solid. MS: 339.0 ([{³⁵Cl, ³⁵Cl}M+H]⁺), 341.0 ([{³⁵Cl, ³⁷Cl}M+H]⁺), ESI pos.

### f) 5-[5-[tert-butyl(dimethyl)silyl]oxy-2-fluoro-phenyl]-6,7-dichloro-1,3-dihydro-1,4-benzodiazepin-2-one

To a solution of 6,7-dichloro-5-(2-fluoro-5-hydroxy-phenyl)-1,3-dihydro-1,4-benzodiazepin-2-one (175 mg, 0.516 mmol) in DMF (1.75 mL) was added at 22° C. imidazole (77.3 mg. 1.14 mmol) followed by tert-butyldimethylchlorosilane (85.5 mg, 0.568 mmol) and the resulting mixture was stirred at 22° C. for 0.5 h. The mixture was concentrated in vacuo. The residue was treated with aqueous NaOH (0.1 M) and extracted with ethyl acetate. The organic layer was washed with aqueous NaOH (0.1 M) and brine successively, dried over sodium sulfate, filtered and concentrated in vacuo. The residue was purified by flash column chromatography (silica, 0-30% ethyl acetate in heptane) followed by crystallization from ethyl acetate/heptane to afford the title compound (122 mg, 52%) as a white solid. MS: 453.2 ([{³⁵Cl, ³⁵Cl}M+H]⁺), 455.1 ([{³⁵Cl, ³⁷Cl}M+H]⁺), ESI pos.

### ¹¹C Radiolabeling Precursor 2

### 5-[5-[tert-butyl(dimethyl)silyl]oxy-2-fluoro-phenyl]-6-chloro-7-methyl-1,3-dihydro-1,4-benzodiazepin-2-one

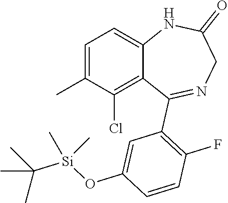 [SMILES: Cc1ccc2c(c1Cl)C(c1cc(O[Si](C)(C)C(C)(C)C)ccc1F)=NCC(=O)N2]

### a) 6-chloro-5-(2-fluoro-5-hydroxy-phenyl)-7-methyl-1,3-dihydro-1,4-benzodiazepin-2-one

To a solution of 6-chloro-5-(2-fluoro-5-methoxy-phenyl)-7-methyl-1,3-dihydro-1,4-benzodiazepin-2-one (500 mg, 1.50 mmol) in DCM (15 mL) was added at −65° C. boron tribromide (1.88 g, 7.51 mmol). The mixture was stirred at −60° C. for 0.5 h. The mixture was then warmed to −20° C. and stirred for 0.5 h. The mixture was quenched with saturated aqueous NaHCO₃ and extracted with ethyl acetate. The organic layer was dried over sodium sulfate, filtered and concentrated in vacuo. The residue was purified by flash column chromatography (silica, 10-100% ethyl acetate in petroleum ether) followed by crystallization from ethyl acetate/heptane to afford the title compound (177 mg, 37%) as a light yellow solid. MS: 319.1 ([{⁷⁹Br, ³⁵Cl}M+H]⁺), 321.1 ([{⁸¹Br, ³⁵Cl or ⁷⁹Br, ³⁷Cl}M+H]⁺), ESI pos.

### b) 5-[5-[tert-Butyl(dimethyl)silyl]oxy-2-fluoro-phenyl]-6-chloro-7-methyl-1,3-dihydro-1,4-benzodiazepin-2-one

To a solution of 6-chloro-5-(2-fluoro-5-hydroxy-phenyl)-7-methyl-1,3-dihydro-1,4-benzodiazepin-2-one (184 mg, 0.577 mol) in DMF (1.8 mL) was added at 22° C. imidazole (86.5 mg, 1.27 mmol) followed by tert-butyldimethylchlorosilane (95.7 mg, 0.635 mmol) and the mixture was stirred at 22° C. for 1 h. The mixture was concentrated in vacuo, treated with aqueous NaOH (0.1 M) and extracted with ethyl acetate. The organic layer was washed with aqueous NaOH (0.1 M) and brine, dried over sodium sulfate, filtered and concentrated in vacuo. The residue was purified by flash column chromatography (silica, 0-45% ethyl acetate in petroleum ether) followed by crystallization from ethyl acetate/heptane to afford the title compound (110 mg, 44%) as a white solid. MS: 433.2 ([{⁷⁹Br, ³⁵Cl}M+H]⁺), 435.2 ([{⁸¹Br, ³⁵Cl or ⁷⁹Br, ³⁷Cl}M+H]⁺), ESI pos.

### EXAMPLES

### Example 1

### 6,7-dichloro-5-(2-fluoro-5-hydroxy-phenyl)-1-methyl-3H-1,4-benzodiazepin-2-one (I)

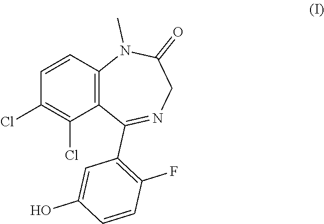 [SMILES: CN1C(=O)CN=C(c2cc(O)ccc2F)c2c1ccc(Cl)c2Cl]

### a) 6,7-dichloro-5-(2-fluoro-5-methoxy-phenyl)-1-methyl-3H-1,4-benzodiazepin-2-one

A solution of 6,7-dichloro-5-(2-fluoro-5-methoxy-phenyl)-1,3-dihydro-1,4-benzodiazepin-2-one (building block A, 120 mg, 0.340 mmol), iodomethane (1.00 g, 7.05 mmol) and potassium carbonate (70 mg, 0.51 mmol) in DMF (3 mL) was stirred at 25° C. for 0.5 h. The reaction was quenched with water and extracted with ethyl acetate. The organic layer was dried over sodium sulfate, filtered and concentrated in vacuo. The residue was purified by preparative HPLC (Phenomenex Synergi C18, 10 μm, 150×25 mm, 0.1% trifluoroacetic acid in water/acetonitrile) to afford the title compound (114 mg, 91%) as a white solid. MS: 367.1 ([{³⁵Cl, ³⁵Cl}M+H]⁺), 369.1 ([{³⁵Cl, ³⁷Cl}M+H]⁺), ESI pos.

### b) 6,7-dichloro-5-(2-fluoro-5-hydroxy-phenyl)-1-methyl-3H-1,4-benzodiazepin-2-one

To a solution of 6,7-dichloro-5-(2-fluoro-5-methoxy-phenyl)-1-methyl-3H-1,4-benzodiazepin-2-one (80 mg, 0.22 mmol) in DCM (9 mL) at 0° C. was added dropwise boron tribromide (273 mg, 1.09 mmol). The reaction mixture was stirred at 0° C. for 1 h, allowed to warm to room temperature and stirred for an additional 5 h. The reaction was quenched with ice-water and extracted with DCM. The organic layer was dried over sodium sulfate, filtered and concentrated in vacuo. The residue was purified by preparative HPLC (Phenomenex Synergi C18, 10 μm, 150×25 mm, 0.225% formic acid in water/acetonitrile) to afford the title compound (59 mg, 77%) as a white solid. MS: 353.1 ([{³⁵Cl, ³⁵Cl}M+H]⁺), 355.1 ([{³⁵Cl, ³⁷Cl}M+H]⁺), ESI pos.

### Example 2

### 6-chloro-5-(2-fluoro-5-hydroxy-phenyl)-1,7-dimethyl-3H-1,4-benzodiazepin-2-one (II)

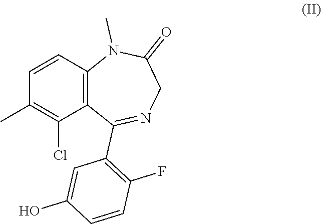 [SMILES: Cc1ccc2c(c1Cl)C(c1cc(O)ccc1F)=NCC(=O)N2C]

### a) 6-chloro-5-(2-fluoro-5-methoxy-phenyl)-1,7-dimethyl-3H-1,4-benzodiazepin-2-one

In analogy to experiment of example 1 a, 6-chloro-5-(2-fluoro-5-methoxy-phenyl)-7-methyl-1,3-dihydro-1,4-benzodiazepin-2-one (building block B) was converted into the title compound (76 mg, 36%) which was obtained as a yellow solid. MS: 347.1 ([{³⁵Cl}M+H]⁺), 349.1 ([{³⁷Cl}M+H]⁺), ESI pos.

### b) 6-chloro-5-(2-fluoro-5-hydroxy-phenyl)-1,7-dimethyl-3H-1,4-benzodiazepin-2-one

In analogy to experiment of example 1 b, 6-chloro-5-(2-fluoro-5-methoxy-phenyl)-1,7-dimethyl-3H-1,4-benzodiazepin-2-one was converted into the title compound (72 mg, 83%) which was obtained as an off-white solid. MS: 333.0 ([{³⁵Cl}M+H]⁺), 335.0 ([{³⁷Cl}M+H]⁺), ESI pos.

### Example [³H]1

### 6,7-Dichloro-5-(2-fluoro-5-hydroxy-phenyl)-1-([³H₃]methyl)-3H-1,4-benzodiazepin-2-one

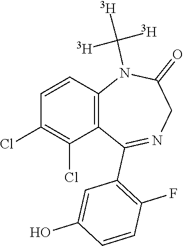 [SMILES: [3H]C([3H])([3H])N1C(=O)CN=C(c2cc(O)ccc2F)c2c1ccc(Cl)c2Cl]

### a) 6,7-Dichloro-5-(2-fluoro-5-methoxy-phenyl)-1-([³H₃]methyl)-3H-1,4-benzodiazepin-2-one

To [³H₃]methyl nosylate (1.85 GBq, 50 mCi, 0.61 μmol) was added a solution of 6,7-dichloro-5-(2-fluoro-5-methoxy-phenyl)-1,3-dihydro-1,4-benzodiazepin-2-one (building block A) (443 μg, 1.25 μmol, 2.0 equiv.) in THF (120 μL). Then a 0.5 M solution of sodium tert-butoxide (6.3 μL, 3.1 μmol, 5.0 equiv.) in THF was added, and the reaction mixture was stirred for 2 h at room temperature. The reaction was quenched by the addition of water (20 μL). The solvent was evaporated under a stream of argon, and the remaining solid was dissolved in MeCN/H₂O 1:1 (180 μL). The crude product was purified by HPLC (Sunfire C18 OBD, 4.6×250 mm, MeCN [A]/H₂O+5% MeCN [B], gradient: 1-18 min 10:90 to 90:10 [A]:[B], 18.0-18.1 90:10 to 95:5, 21.0 to 21.1 95:5 to 10:90, run time 24 min, flow rate 1 mL/min, 236 nm, oven temperature 40° C.; 5 injections). The pure fractions were combined, frozen, and lyophilized under vacuum for 1.5 h. The product was used directly in the next step without further characterization.

### b) 6,7-Dichloro-5-(2-fluoro-5-hydroxy-phenyl)-1-([³H₃]methyl)-3H-1,4-benzodiazepin-2-one

6,7-Dichloro-5-(2-fluoro-5-methoxy-phenyl)-1-([³H₃]methyl)-3H-1,4-benzodiazepin-2-one (expected from previous experiment: 131 μg, 0.35 μmol, 1 equiv., 28 mCi) was dissolved in dichloromethane (0.5 mL) and transferred to a 1-mL Alltech tube. The solvent was evaporated under a stream of Argon. This was repeated three times with in total 1.5 mL of dichloromethane. The residue was dissolved in dichloromethane (extra dry, over molecular sieves, 0.15 mL) and treated with a IM solution of borontribromide (5.3 μL, 5.3 μmol, 15 equiv.). The tube was closed with a teflon-sealed plastic cap. The solution turned yellow and was stirred for 4 h at 40° C. (oil bath temperature. Radio-HPLC analysis revealed almost complete consumption of the starting material. At room temperature, the reaction was quenched by the addition of water (20 μL). The solvent was evaporated under a stream of Argon, and the remaining solid was dissolved in MeCN/H₂O 1:1 (150 μL). The crude product was purified by HPLC (Sunfire C18 OBD, 4.6×250 mm, MeCN [A]/H₂O+5% MeCN [B], gradient: 1-18 min 10:90 to 90:10 [A]:[B], 18.0-18.1 90:10 to 95:5, 21.0 to 21.1 95:5 to 10:90, run time 24 min, flow rate 1 mL/min, 236 nm, oven temperature 40° C.; 5 injections). The pure fractions were combined, frozen and lyophilized under vacuum for 2 h. The pure tritium-labeled compound (337 MBq, 9.1 mCi) was dissolved and stored in ethanol (10 mL). The radiochemical purity of 96% was determined by radio-HPLC and the specific activity of 3.0 TBq/mmol (81 Ci/mmol) by mass spectrometry (MS). The identity of the labeled compound was confirmed by HPLC (by co-injecting the unlabeled reference standard) and by MS. MS: m/z=353.0 [M(H)+H]⁺ (3%), 355.0 [M(³H)+H]⁺ (0%), 357.0 [M(³H₂)+H]⁺ (6%), 359.0.1 [M(³H₃)+H]⁺ (90%).

### Example [³H]2

### 6-Chloro-5-(2-fluoro-5-hydroxy-phenyl)-7-methyl-1-([³H₃]methyl)-3H-1,4-benzodiazepin-2-one

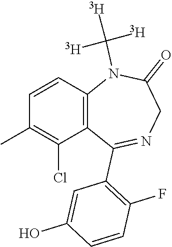 [SMILES: [3H]C([3H])([3H])N1C(=O)CN=C(c2cc(O)ccc2F)c2c1ccc(C)c2Cl]

### a) 6-Chloro-5-(2-fluoro-5-methoxy-phenyl)-7-methyl-1-([³H₃]methyl)-3H-1,4-benzodiazepin-2-one

To [³H₃] methyl nosylate (1.85 GBq, 50 mCi, 0.61 μmol) was added a solution of 6-chloro-5-(2-fluoro-5-methoxy-phenyl)-7-methyl-1,3-dihydro-1,4-benzodiazepin-2-one (building block B) (417 μg, 1.25 μmol, 2.0 equiv.) in THF (120 μL). Then a 0.5 M solution of sodium tert-butoxide (6.3 μL, 3.1 μmol, 5 equiv.) in THF was added, and the reaction mixture was stirred for 130 min at room temperature. The reaction was quenched by the addition of water (20 μl). The solvent was evaporated under a stream of argon, and the remaining solid was dissolved in MeCN/H₂O 1:1 (160 μL). The crude product was purified by HPLC (Sunfire C18 OBD, 4.6×250 mm, MeCN [A]/H₂O+5% MeCN [B], gradient: 1-18 min 10:90 to 90:10 [A]:[B], 18.0-18.1 90:10 to 95:5, 21.0 to 21.1 95:5 to 10:90, run time 24 min, flow rate 1 mL/min, 236 nm, oven temperature 40° C.; 5 injections) The pure fractions were combined, frozen, and lyophilized under vacuum for 2 h. The pure tritium-labeled compound (1040 MBq, 28.1 mCi) was dissolved and stored in ethanol (10 mL) until further use. The radiochemical purity of >99% was determined by radio-HPLC.

### b) 6-Chloro-5-(2-fluoro-5-hydroxy-phenyl)-7-methyl-1-([³H₃]methyl)-3H-1,4-benzodiazepin-2-one

The solution of 6-chloro-5-(2-fluoro-5-methoxy-phenyl)-7-methyl-1-([³H₃]methyl)-3H-1,4-benzodiazepin-2-one (124 μg, 0.351 μmol, 1 equiv., 28.1 mCi) in ethanol (10 mL) was concentrated to dryness at the Rotavap at 40° C. The residue was dissolved in dichloromethane (0.5 mL) and transferred to a 1-mL Alltech tube. The solvent was evaporated under a stream of Argon. This was repeated three times with in total 1.5 mL of dichloromethane. The residue was dissolved in dichloromethane (extra dry, over molecular sieves, 0.15 mL) and treated with a 1M solution of borontribromide (3.5 μL, 3.5 μmol, 10 equiv.). The tube was closed with a teflon-sealed plastic cap. The solution turned yellow and was stirred for 3 h at 40° C. (oil bath temperature). Radio-HPLC analysis revealed almost complete consumption of the starting material. At room temperature, the reaction was quenched by the addition of water (20 μL). The solvent was evaporated under a stream of Argon and the remaining solid dissolved in MeCN/H₂O 1:1 (200 μL). The crude product was purified by HPLC (Sunfire C18 OBD, 4.6×250 mm, MeCN [A]/H₂O+5% MeCN [B], gradient: 1-18 min 10:90 to 90:10 [A]:[B], 18.0-18.1 90:10 to 95:5, 21.0 to 21.1 95:5 to 10:90, run time 24 min, flow rate 1 mL/min, 236 nm, oven temperature 40° C.; 5 injections) The pure fractions were combined, frozen and lyophilized under vacuum for 2 h. The pure tritium-labeled compound (410.7 MBq, 11.1 mCi) was dissolved and stored in ethanol (10 mL). The radiochemical purity of 97% was determined by radio-HPLC and the specific activity of 3.1 TBq/mmol (83 Ci/mmol) by mass spectrometry (MS). The identity of the labeled compound was confirmed by HPLC (by co-injecting the unlabeled reference standard) and by MS. MS: m/z=333.1 [M(H)+H]⁺ (2%), 335.1 [M(³H)+H]⁺ (0%), 337.1 [M(³H₂)+H]⁺ (7%), 339.1 [M(³H₃)+H]⁺ (91%).

### Example [¹¹C]1

### [¹¹C]6,7-Dichloro-5-(2-fluoro-5-hydroxy-phenyl)-1-methyl-3H-1,4-benzodiazepin-2-one

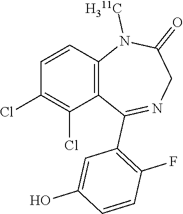 [SMILES: [11CH3]N1C(=O)CN=C(c2cc(O)ccc2F)c2c1ccc(Cl)c2Cl]

The radiochemical synthesis of this tracer proceeded as [¹¹C]6-chloro-5-(2-fluoro-5-hydroxy-phenyl)-1,7-dimethyl-3H-1,4-benzodiazepin-2-one (Example [¹¹C]2) except the preparative mobile phase flow rate was 15 mL/min. The preparative retention time of the radiotracer was 6.1 min. Quality control of this radiotracer was done the same as below except the mobile phase was 35:65 acetonitrile (MeCN)/TEA buffer (pH 7.2) and the UV wavelength monitored was 254 nm. Comparable radiochemical and chemical purity, specific activity (molar activity), and chemical identity results were obtained.

### Example [¹¹C]2

### [¹¹C]6-Chloro-5-(2-fluoro-5-hydroxy-phenyl)-1,7-dimethyl-3H-1,4-benzodiazepin-2-one

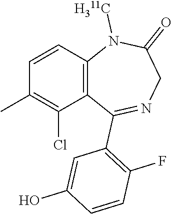 [SMILES: Cc1ccc2c(c1Cl)C(c1cc(O)ccc1F)=NCC(=O)N2[11CH3]]

A standard gas carbon dioxide target of a General Electric (GE) Medical Systems (GEMS, Uppsala, Sweden) PETTrace cyclotron was filled with high purity nitrogen containing 0.5% oxygen. The target was irradiated with a proton beam at 60 μA for 25 min to produce approximately 2-3 Ci (74-111 GBq) of [¹¹C]carbon dioxide. The radioactive gas was transferred to a GE FXMeI module that synthesizes ¹¹CH₃I in approximately 10 min, after which the ¹¹CH₃I was transferred by helium gas to an appropriate hot cell for radiosynthesis. The precursor 5-[5-[tert-butyl(dimethyl)silyl]oxy-2-fluoro-phenyl]-6-chloro-7-methyl-1,3-dihydro-1,4-benzodiazepin-2-one (1±0.3 mg) was dissolved in 200 μL of dimethylformamide (DMF) and added to a vial containing potassium carbonate (1±0.3 mg) that was subsequently sealed. Prior to the end of bombardment (EOB), the vial was placed in a lead-lined synthesis cell. After trapping of ¹¹CH₃I, the vial was heated (80° C.) for 3 min. Hydrochloric acid (1 mL) was added to the reaction mixture and the vial was heated (80° C.) for 1 min. The reaction solution was diluted with 1 mL of 30% acetonitrile:70% aqueous buffer (57 mM TEA adjusted to pH 7.2 with o-phosphoric acid) and injected onto the semipreparative HPLC column (XBridge C-18, 10 μm, 10 mm×150 mm), eluting with 30% acetonitrile:70% aqueous buffer (57 mM TEA adjusted to pH 7.2 with o-phosphoric acid) at 10 mL/min with the effluent monitored for radioactivity content and UV (254 nm). The product peak (t_{R}=6.7 min, k′=5.7) was collected in 50 mL of water. The product solution was eluted onto a conditioned Waters C18 SepPak Plus (Waters Corp.), and the SepPak was washed with water HPLC water (10 mL). The radiotracer product was eluted from the SepPak with absolute ethanol (1 mL) followed by sterile saline (10 mL) through a 0.2 μm sterile Millipore FG filter (25 mm) into a sterile product vial preloaded with sterile saline (4 mL). Aliquots were removed from the final product vial for quality control analysis.

Analytical HPLC was performed to determine radiochemical and chemical purity, specific activity (molar activity), and chemical identity using an XBridge C-18 column (3.5 μm, 4.6 mm×100 mm) eluted with 30:70 acetonitrile (MeCN)/TEA buffer (pH 7.2), eluted at 2 mL/min, and monitored at 236 nm. The final radiotracer product demonstrated a radiochemical purity of greater than 95% with an average yield over 100 mCi of product which co-eluted with authentic cold reference. The average specific activity (molar activity) was greater than 10 Ci per micromole (370 GBq per micromole).

### Example 3—Assay Procedures

### Membrane Preparation and Binding Assay for γ1-Containing GABA_{A} Subtypes

The affinity of compounds of formula (I) and (II) (see Examples 1 and 2 above) at GABA_{A} γ1 subunit-containing receptors was measured by competition for [³H]RO7239181 (67.3 Ci/mmol; Roche; described, e.g., in WO2021198124) binding to membranes from HEK293F cells (ThermoFisher R79007) expressing human (transiently transfected) receptors of composition α5β2γ1, α2β2γ1, α1β2γ1. For better protein expression of the α2 subunit-containing receptors, the 28 amino acid long signal peptide (Met1 to Ala28) of the human GABA_{A} α2 subunit was substituted by the 31 amino acid long signal peptide (Met1 to Ser31) of human GABA_{A} α5 subunit.

Harvested pellets from HEK293F cells expressing the different GABA_{A} receptor subtypes were resuspended in Mannitol Buffer pH 7.2-7.4 (Mannitol 0.29M, Triethylamine 10 mM, Acetic acid 10 mM, EDTA 1 mM plus protease inhibitors (20 tablets Complete, Roche Diagnostics Cat. No. 05 056 489 001 per liter)), washed two times and then resuspended at 1:10 to 1:15 dilution in the same buffer. Cell disruption was performed by stirring the suspension in a Parr vessel #4637 at 435 psi for 15 minutes, and then the suspensions were centrifuged at 1000×g for 15 minutes at 4° C. (Beckman Avanti J-HC; rotor JS-4.2). The supernatant (S1) was transferred in a 2 l Schott flask and the pellet (P1) was resuspended with Mannitol Buffer up to 175 ml. The resuspended pellet was transferred into a 250 ml Corning centrifugal beaker and centrifuged at 1500×g for 10 minutes at 4° C. (Beckman Avanti J-HC; rotor JS-4.2). The supernatant (S1) was then transferred in the 2 l Schott flask and the pellet was discarded. The supernatants (S1) were centrifuged in 500 ml Beckman polypropylene centrifugal beaker at 15000×g for 30 minutes at 4° C. (Beckman Avanti J-20 XP; rotor JLA-10.500). The pellet (P2) was resuspended with Mannitol Buffer 1:1 and frozen at −80° C. The supernatant (S2) was centrifuged in 100 ml Beckman polypropylene centrifugal tubes at 48000×g for 50 minutes at 4° C. (Beckman Avanti J-20 XP; rotor JA-18). The supernatant (S3) was discarded and the pellet (P3) was resuspended with 1:1 Mannitol Buffer. The P2 and P3 protein concentration was determined with the BIORAD Standard assay method with bovine serum albumin as standard and measured on the NANO-Drop 1000. The membrane suspension was aliquots (500 μl per tube) and stored at −80° C. until required.

Membrane homogenates were resuspended and polytronised (Polytron PT1200E Kinematica AG) in Potassium Phosphate 10 mM, KCl 100 mM binding buffer at pH 7.4 to a final assay concentration determined with a previous experiment.

Radioligand binding assays were carried out in a volume of 200 μL (96-well plates) which contained 100 μL of cell membranes, [³H]RO7239181 at a concentration of 1.5 nM (α5β2γ1) or 20-30 nM (α1β2γ1, α2β2γ1) and the test compound in the range of [0.3-10000]×10⁻⁹ M. Nonspecific binding was defined by 10×10⁻⁶ (α5β2γ1) and 30×10⁻⁶ M RO7239181 and typically represented less than 5% (α5β2γ1) and less than 20% (α1β2β1, α2β2γ1) of the total binding. Assays were incubated to equilibrium for 1 hour at 4° C. and then, membranes were filtered onto unifilter (96-well white microplate with bonded GF/C filters preincubated 20-50 minutes in 0.3% Polyethylenimine) with a Filtermate 196 harvester (Packard BioScience) and washed 4 times with cold Potassium Phosphate 10 mM pH 7.4, KCl 100 mM binding buffer. After anhydrousing, filter-retained radioactivity was detected by liquid scintillation counting. Kᵢ values were calculated using Excel-Fit (Microsoft) and are the means of two determinations.

The compounds of the accompanying examples were tested in the above described assays, and the preferred compounds were found to possess a Kᵢ value for the displacement of [³H]RO7239181 from GABA_{A} γ1 subunit-containing receptors (e.g. α5β2γ1, α2β2γ1, α1β2γ1) of 100 nM or less. Most preferred are compounds with a Kᵢ (nM)<50. Representative test results, obtained by the above described assay measuring binding affinity to HEK293 cells expressing human (h) receptors, are shown in the Table 1.

### Membrane Preparation and Binding Assay for γ2-Containing GABA_{A} Subtypes

The affinity of compounds at GABA_{A} γ2 subunit-containing receptors was measured by competition for [³H]Flumazenil (81.1 Ci/mmol; Roche) binding to HEK293F cells expressing human (transiently transfected) receptors of composition α1β3γ2.

Harvested pellets from HEK293F cells expressing the different GABA_{A} γ2 receptor subtypes were resuspended in Mannitol Buffer pH 7.2-7.4 and processed as described above for the cells expressing the GABA_{A} γ1 subunit-containing receptors.

Radioligand binding assays were carried out in a volume of 200 μL (96-well plates) which contained 100 μL of cell membranes, [³H]Flumazenil at a concentration of 1 nM and the test compound in the range of [0.1·10⁻³−10]×10⁻⁶ M. Nonspecific binding was defined by 10⁻⁵ M Diazepam and typically represented less than 5% of the total binding. Assays were incubated to equilibrium for 1 hour at 4° C. and harvested onto GF/C uni-filters (Packard) by filtration using a Packard harvester and washing with ice-cold wash buffer (50 mM Tris; pH 7.5). After anhydrousing, filter-retained radioactivity was detected by liquid scintillation counting. Kᵢ values were calculated using Excel-Fit (Microsoft) and are the means of two determinations.

The compounds of the accompanying examples were tested in the above described assay, and the preferred compounds were found to possess large Kᵢ value for displacement of [³H]Flumazenil from the α1β3γ2 subtype of the human GABA_{A} receptor of 100 nM or above. Most preferred are compounds with a Kᵢ α1β3γ2 (nM)>300. In a preferred embodiment the compounds of the invention are binding selectively for the γ1 subunit-containing GABA_{A} receptors relative to γ2 subunit-containing GABA_{A} receptors. In particular, compounds of the present invention have γ2/γ1 selectivity ratio defined as “Kᵢ α1β3γ2 (nM)/Kᵢ α2β2γ1 (nM)” above 10-fold, or LogSel defined as “Log[Kᵢ 1β3γ2 (nM)/Kᵢ α2β2γ1 (nM)]” above 1. Representative test results, obtained by the above described assay measuring binding affinity to HEK293 cells expressing human (h) receptors, are shown in the Table 1 below.

TABLE 1

| | Ki h- | Ki h- | Ki h- | Ki h- | γ2/γ1 | |
|---|---|---|---|---|---|---|
| | GABA_{A} | GABA_{A} | GABA_{A} | GABA_{A} | Selec- | |
| Exam- | α5β2γ1 | α2β2γ1 | α1β2γ1 | α1β3γ2 | tivity | |
| ple | (nM) | (nM) | (nM) | (nM) | Ratio | LogSel |
| 1 | 0.34 | 2.27 | 2.90 | 28.7 | 12.7 | 1.10 |
| 2 | 0.53 | 1.49 | 3.49 | 58.2 | 39.0 | 1.59 |

### Functional Expression of GABA_{A} Receptors

### Xenopus Oocytes Preparation

Xenopus laevis oocytes at maturation stages V-VI were used for the expression of cloned mRNA encoding GABA_{A} receptor subunits. Oocytes ready for RNA micro-injection were bought from Ecocyte, Castrop-Rauxel, Germany and stored in modified Barth's medium (composition in mM: NaCl 88, KCl 1, NaHCO₃ 2.4, HEPES 10, MgSO₄ 0.82, CaNO₃ 0.33, CaCl₂ 0.33, pH=7.5) at 20° C. until the experiment.

### Xenopus Oocytes Microinjection

Oocytes were plated in 96-well plates for microinjection using the Roboinject automated instrument (MultiChannelSystems, Reutlingen, Germany). Approximately 50 nL of an aqueous solution containing the RNA transcripts for the subunits of the desired GABA_{A} receptor subtype was injected into each oocyte. RNA concentrations ranged between 20 and 200 pg/μL/subunit and were adjusted in pilot experiments to obtain GABA responses of a suitable size and a maximal effect of Flunitrazepam, Triazolam and Midazolam, reference benzodiazepine positive allosteric modulators (PAM) at the GABA_{A} receptor benzodiazepine (BZD) binding site. Oocytes were kept in modified Barth's medium (composition in mM: NaCl 88, KCl 1, NaHCO₃ 4, HEPES 10, MgSO₄ 0.82, CaNO₃ 0.33, CaCl₂ 0.33, pH=7.5) at 20° C. until the experiment.

### Electrophysiology

Electrophysiological experiments were performed using the Roboocyte instrument (MultiChannelSystems, Reutlingen, Germany) on days 3 to 5 after the micro-injection of mRNA. During the experiment the oocytes were constantly superfused by a solution containing (in mM) NaCl 90, KCl 1, HEPES 5, MgCl₂ 1, CaCl₂ 1 (pH 7.4). Oocytes were impaled by two glass microelectrodes (resistance: 0.5-0.8 MΩ) which were filled with a solution containing KCl 1M+K-acetate 1.5 M and voltage-clamped to −80 mV. The recordings were performed at room temperature using the Roboocyte two-electrode voltage clamp system (Multichannelsystem). After an initial equilibration period of 1.5 min GABA was added for 1.5 min at a concentration evoking approximately 20% of a maximal current response (EC₂₀). After another rest interval of 2.5 min GABA was again added evoking a response of similar amplitude and shape. 0.5 min after the onset of this second GABA application the test compound, at a concentration corresponding to approximatively 30-fold its Kᵢ α2β2γ1, was added while GABA was still present. Current traces were recorded at a digitization rate of 10 Hz during and shortly before and after the GABA application.

Each compound and concentration was tested on at least 3 oocytes. Different oocytes were used for different compound concentrations. The reference PAMs, Flunitrazepam, Triazolam and Midazolam, potentiated the GABA-induced current in α2β2γ1 GABA_{A} receptor subtype expressing oocytes by approximatively 60%.

### Data Analysis

For the analysis, the digitized current traces of the first and second GABA response were superimposed and, if necessary, rescaled to equal maximal amplitudes. The ratio between the two responses during the time interval of test compound application was calculated point by point. The extremum of the resulting “ratio trace” was taken as the efficacy (“Fold increase”) of the compound expressed as “% modulation of GABA EC₂₀” (100*(Fold increase−1)).

The results are shown in Table 2.

TABLE 2

| | Ki h-GABA_{A} | Fold increase h-GABA-A | Efficacy |
|---|---|---|---|
| Example | α2β2γ1 (nM) | α2β2γ1 oocyte @ 30-fold Ki | (GABA)% |
| 1 | 2.27 | 1.90 | 90 |
| 2 | 1.49 | 1.70 | 70 |

### Example 4—Reference Compounds

Benzodiazepines reference compounds (classical marketed benzodiazepines) and their structural analogues listed below were tested for their affinity towards the GABA_{A} receptor α1β2γ1 and α2β2γ1 subtypes as well as in the GABA_{A} receptor α1β3γ2 subtype. The results are shown in Table 3.

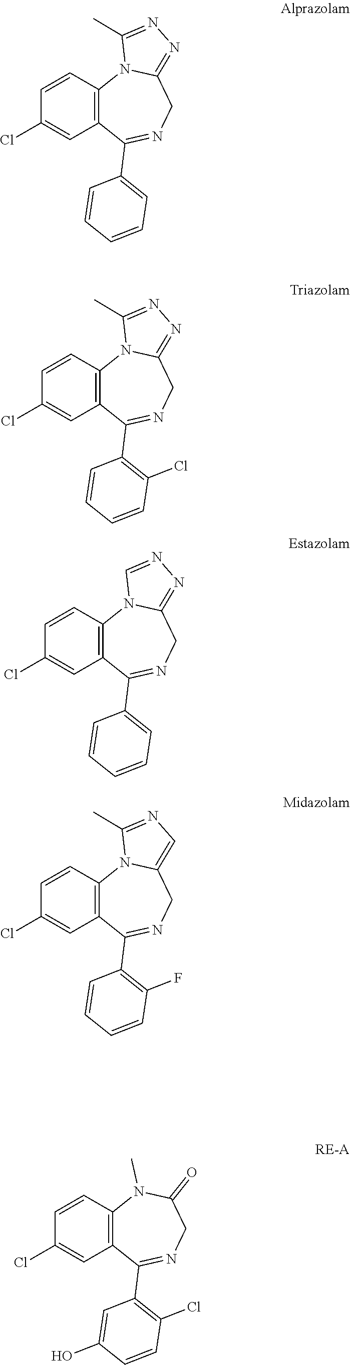

TABLE 3

| | Ki | Ki | Ki | | |
|---|---|---|---|---|---|
| | h-GABA_{A} | h-GABA_{A} | h-GABA_{A} | γ2/γ1 | |
| | α1β2γ1 | α2β2γ1 | α1β3γ2 | Selectivity | |
| Example | (nM) | (nM) | (nM) | Ratio | LogSel |
| Alprazolam | 5923 | 3945 | 19.6 | 0.0050 | −2.3 |
| Triazolam | 44.2 | 46.2 | 1.5 | 0.032 | −1.5 |
| Estazolam | ND | 3182 | 28.4 | 0.0089 | −2.0 |
| Midazolam | 1153.2 | 737.7 | 5.0 | 0.0068 | −2.2 |
| RE-A | 6.84 | 2.79 | 1.5 | 0.54 | −0.27 |
| Example 1 | 2.90 | 2.27 | 28.7 | 12.7 | 1.1 |

### Example 5—In Vitro Autoradiography

Autoradiographical analyses of [³H]-(I) and [³H]-(II) were performed with coronal brain sections from GABA_{A} γ1 receptor knockout mice (C57BL/6NTac-Gabrg1) and wild-type controls. Tissue sections (10 μm) were cut in a cryostat, thaw-mounted on microscope glass slides and incubated in incubation buffer (50 mM Tris-HCl, pH 7.4) containing 0.3 nM radioligand (molar activity 83 Ci/mmol and 81 Ci/mmol for [³H]-(II) and [³H]-(I), respectively) at room temperature for 30 minutes. After incubation, all sections were rinsed three times in ice-cold wash buffer (50 mM Tris-HCl, pH 7.4) for 10 minutes and dipped three times in distilled water at 4° C. Slide-mounted brain sections were dried in a ventilated fridge for at least 2 hours and exposed to a Fuji Imaging Plate for 5 days. The imaging plate was scanned at a resolution of 25 μm in a Fujifilm high-resolution plate scanner. Visualization and quantification of autoradiographies was performed by the MCID™ image analysis program.

### Results

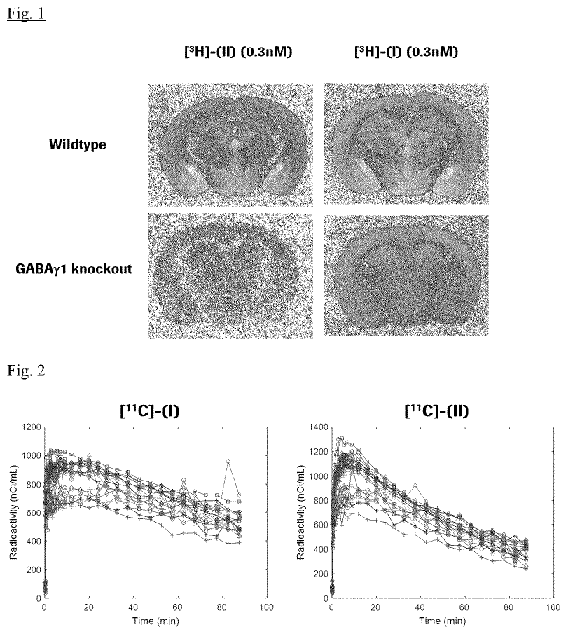

Autoradiograms revealed a distribution binding pattern in line with the expected enriched expression of the GABA_{A} γ1 receptor subtype in limbic brain regions such as the amygdala (FIG. 1). Radioligand binding was clearly reduced in brain sections from GABA_{A} γ1 receptor knockout mice. [³H]-(II) showed 97% specific binding in the amygdala, while [³H]-(I) revealed 65% specific binding.

### Example 6—In Vivo PET Scans

PET imaging experiments were performed in male papio anubis olive brown baboons to examine the characteristics of [¹¹C]-(I) and [¹¹C]-(II) in vivo. The final radiotracer product was radiochemically pure (>95%) up to 40 min post its end of synthesis with an average final calculated specific activity of >555 GBq/μmol. The PET camera used was the Siemens HRRT with an in-plane field-of-view (FOV) of 30 cm and an axial FOV of 24 cm. Each dynamic PET scan started with an intravenous bolus injection of the radiotracer (approx. 700 MBq) and continued for 90 min in a 3D list mode. A set of volumes of interest (VOIs) for 16 brain regions were defined on MRIs of individual animals referring to a standard VOI template. VOIs were transferred to PET space using the coregistration parameters to generate time-activity curves (TACs) of brain regions.

### Results

Upon intravenous injection in baboons, both PET tracers showed rapid initial brain uptake (peaks before or around 10 min in most regions, but slower peaks in lower peak regions) and gradual washout as visible in FIG. 2. [¹¹C]-(II) had earlier peaks than [¹¹C]-(I) in general, suggestive of faster entrance to the brain. Slower clearance of [¹¹C]-(I) suggested slower dissociation or more likely higher non-specific binding for this tracer. Altogether, both tracer candidates demonstrated good transport across the blood-brain barrier, low non-specific retention, and appropriate clearance kinetics. Collectively, these properties render both radiotracers a promising PET imaging agent for the visualization of the GABA_{A} γ1 receptor subtype.

1. A compound of formula (I) or (II) 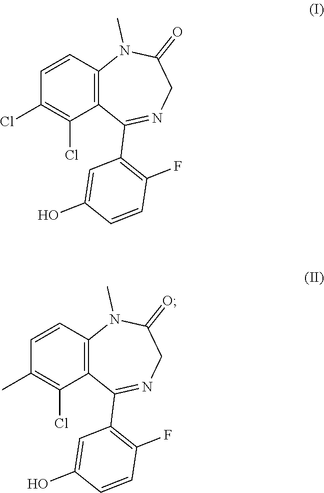 [SMILES: CN1C(=O)CN=C(c2cc(O)ccc2F)c2c1ccc(Cl)c2Cl ; Cc1ccc2c(c1Cl)C(c1cc(O)ccc1F)=NCC(=O)N2C]
or a pharmaceutically acceptable salt thereof, wherein said compound comprises a radiolabel.

2. The compound of formula (I) or (II) according to claim 1, wherein said radiolabel is selected from ¹¹C and ³H.

3. The compound of formula (I) or (II) according to claim 2, selected from the group consisting of 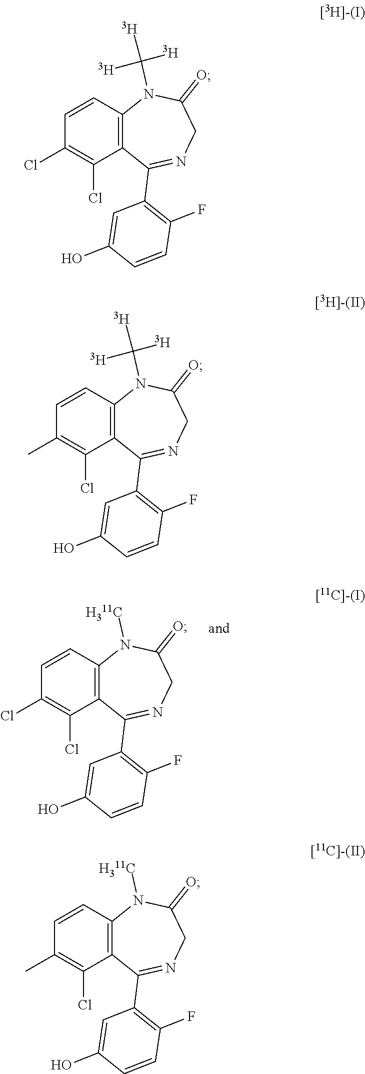 [SMILES: [3H]C([3H])([3H])N1C(=O)CN=C(c2cc(O)ccc2F)c2c1ccc(Cl)c2Cl ; [3H]C([3H])([3H])N1C(=O)CN=C(c2cc(O)ccc2F)c2c1ccc(C)c2Cl ; [11CH3]N1C(=O)CN=C(c2cc(O)ccc2F)c2c1ccc(Cl)c2Cl ; Cc1ccc2c(c1Cl)C(c1cc(O)ccc1F)=NCC(=O)N2[11CH3]]
or a pharmaceutically acceptable salt thereof.

4. The compound of formula (I) or (II) according to claim 3, which is 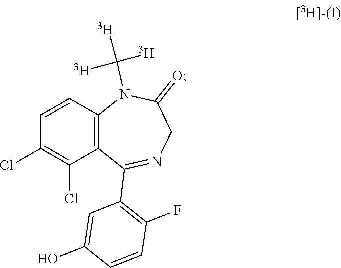 [SMILES: [3H]C([3H])([3H])N1C(=O)CN=C(c2cc(O)ccc2F)c2c1ccc(Cl)c2Cl]
or a pharmaceutically acceptable salt thereof.

5. The compound of formula (I) or (II) according to claim 3, which is 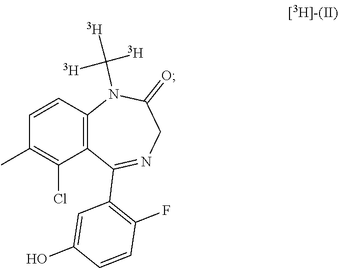 [SMILES: [3H]C([3H])([3H])N1C(=O)CN=C(c2cc(O)ccc2F)c2c1ccc(C)c2Cl]
or a pharmaceutically acceptable salt thereof.

6. The compound of formula (I) or (II) according to claim 3, which is 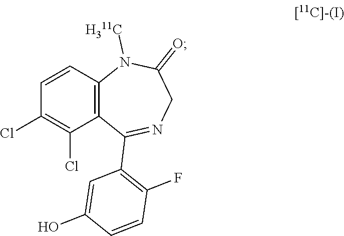 [SMILES: [11CH3]N1C(=O)CN=C(c2cc(O)ccc2F)c2c1ccc(Cl)c2Cl]
or a pharmaceutically acceptable salt thereof.

7. The compound of formula (I) or (II) according to claim 3, which is 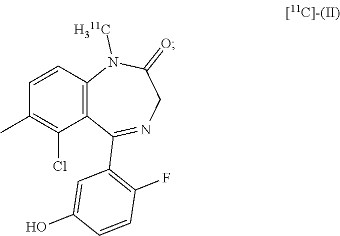 [SMILES: Cc1ccc2c(c1Cl)C(c1cc(O)ccc1F)=NCC(=O)N2[11CH3]]
or a pharmaceutically acceptable salt thereof.

8. A method of diagnostic imaging of GABA_{A} γ1 in a mammal, comprising:
(a) administering to the mammal a detectable quantity of a radiolabeled compound according to acclaim 1, or of a pharmaceutically acceptable salt thereof, and
(b) detecting the radiolabeled compound when associated with GABA_{A} γ1.

9. The method according to claim 8, wherein said detecting is done via autoradiography and/or positron-emission tomography (PET).
